(19) United States
(12) Patent Application Publication
(10) Pub. No.: US 2017/0071165 A1
(43) Pub. Date: Mar. 16, 2017

(54) KITS AND METHODS FOR MEASURING THE IMPACT OF A DIET

(71) Applicant: Mars, Incorporated, McLean, VA (US)

(72) Inventor: Susan Hart Chapman, West Chester, OH (US)

(73) Assignee: Mars, Incorporated, McLean, VA (US)

(21) Appl. No.: 15/360,623

(22) Filed: Nov. 23, 2016

Related U.S. Application Data
(63) Continuation of application No. 13/217,397, filed on Aug. 25, 2011.
(60) Provisional application No. 61/377,476, filed on Aug. 27, 2010.
(60) Provisional application No. 61/377,478, filed on Aug. 27, 2010.
(60) Provisional application No. 61/377,479, filed on Aug. 27, 2010.

(51) Int. Cl. A01K 29/00; A61K 49/00; A61K 9/00; A61B 5/11

(52) U.S. Cl. CPC A01K 29/005; A61B 5/1124; A61K 49/0004; A61K 9/0056

(57) ABSTRACT

Embodiments described herein provide for a method of assessing the health of a companion animal. The method can include providing a first group of companion animals, the first group comprising a first plurality of companion animals; subjecting each of the first plurality of companion animals to a performance assessment; evaluating a performance of each companion animal based on a plurality of attributes, wherein the attributes are associated with the health of the companion animal, wherein the evaluating can include providing a performance profile; accounting for the attributes exhibited by each companion animal during the performance; assigning a performance index based on the performance profile given the attributes accounted for.

### CROSS-REFERENCE TO RELATED APPLICATIONS

This application is a continuation of U.S. application Ser. No. 13/217,397, filed on Aug. 25, 2011 (pending), which, in turn, claims the benefit of U.S. Provisional Application No. 61/377,476, filed Aug. 27, 2010, U.S. Provisional Application No. 61/377,478, filed on Aug. 27, 2010, and US. Provisional Application No. 61/377,479, filed on Aug. 27, 2010, all of which are hereby incorporated by reference herein in their entirety.

### FIELD

Methods are provided for assessing the health of animals. The methods specifically relate to assessing the health of an animal by evaluating the performance of the animal subjected to a physical assessment.

### BACKGROUND

An increasing number of people are acquiring and caring for a great variety of companion animals. Many companion animal breeders, owners, and caregivers would like their companion animals to live longer and healthier lives. Breeders, owners, and caregivers of these companion animals have a desire to understand the physical and biological attributes, genetic makeup, heritable disease, disorder background, and longevity of their companion animals. While companion animals and other animals generally live longer and have a better quality of life today due to improved nutrition and medical care, substantial investments in time, effort, and financial resources are made to characterize the health state of those companion animals. Periodic health assessments of those companion animals are also desirable.

It would be of value to provide a method for assessing the health of a companion animal. Many indicators of health and wellness in companion animals exist, including observation of the animal. An example of an indicator of the health and wellness of a companion animal is the mobility of the companion animal. A companion animal may have an expected mobility based on the companion animal's chronological age, but the actual mobility of the companion animal may vary from the expected mobility as the companion animal ages. This variance may be a result of any number of factors, such as, but not limited to, activity level, weight management, disease, arthritic conditions, etc. The observation of the actual mobility of the companion animal can be utilized to assess the overall health and wellness of the animal, and it can be used to assess specific health areas of the animal, such as mobility. Based on the overall health or the specific health area, individualized programs or interventions could then be recommended for the companion animal. Such a program or intervention could maintain, enhance, or improve the companion animal's health through lifestyle changes such as dietary modification, supplement administration, weight loss/management plans, physical activity recommendations, veterinary intervention, and combinations thereof. Accordingly, it would be beneficial to provide a method for assessing the health of the animal.

### SUMMARY

Methods and kits for assessing the performance of a companion animal in performing physical tasks, such as routine daily tasks, are provided that allow an owner, handler, or anyone to gauge the health or wellness of the animal. This assessment can be performed on any of the health attributes of companion animals. Additionally, with the increasing availability of nutrition, such as pet foods or supplements, directed toward increasing the health or wellness of companion animals, such an assessment becomes even more practical when provided with such foods or supplements. When so provided, these assessments can be used to evaluate the effectiveness of nutrition, especially in a comparative setting, and its impact on the health of wellness of companion animals. Such assessments could thus be beneficial and are provided by certain embodiments.

In one embodiment, a kit is provided comprising a pet food formulated to provide a health benefit, a credentialing tool comprising a performance profile and instructions for feeding the food and assessing the impact thereof on the health of the pet using the credentialing tool. The performance profile comprises a compilation of past performance assessments for pets of a similar age, size and/or breed.

In a further embodiment, a method for assessing the impact of a diet on a pet is provided. The method comprises feeding the pet the diet, and periodically assessing the impact of the diet by comparing the pet's performance of at least one activity to a performance profile comprising a compilation of performance assessments for pets of a similar age, size and/or breed.

### BRIEF DESCRIPTION OF THE DRAWINGS

The description provided herein may be further described and understood via reference to the accompanying drawings.

FIG. 1 depicts one embodiment of a stairs apparatus.

FIG. 2 depicts one embodiment of a rails apparatus.

FIG. 3 depicts a correlation of a Visual Analog Score to frequency of attributes for one embodiment.

FIG. 4 depicts a correlation of a Visual Analog Score to frequency of attributes for one embodiment.

FIG. 5 depicts a correlation of a Visual Analog Score to frequency of attributes for one embodiment.

In accordance with common practice, the various described features are not drawn to scale but are drawn to emphasize specific features relevant to the examples. Like reference numbers and designations in the various drawings indicate like elements.

### DETAILED DESCRIPTION

Definitions

As used herein, the articles including “the”, “a”, and “an”, when used in a claim or in the specification, are understood to mean one or more of what is claimed or described.

As used herein, the terms “include”, “includes”, and “including” are meant to be non-limiting.

As used herein, the term “plurality” means more than one.

As used herein, the terms “animal” or “pet” mean a domestic animal including, but not limited to domestic dogs, cats, horses, cows, ferrets, rabbits, pigs, rats, mice, gerbils, hamsters, horses, and the like. Domestic dogs and cats are particular examples of pets and are referred to herein as “companion animals.” It should be understood that throughout this disclosure when using the term animal, pet, or companion animal, the animal, pet, or companion animal is in a non-diseased state, unless otherwise stated.

As used herein, the terms “ability to move” mean the ability of the animal to perform any of a number of multitude of tasks, such as the activities of daily living described herein, like standing from rest, walking, running, climbing, traversing rails, jumping, among other things. In general, the more the effort that is needed means that the animal has less ability. Other expressions for describing the ability to move include the extent to be able to move and the capability to move.

As used herein, the term “activity” means how active the animal is in a period of time. Animals can be more or less active based on how active the animal is in a period of time.

As used herein, the term “mobility” means how well the animal moves, which can be measured as known to those of ordinary skill in the art and as referenced in the patent publications herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” . . . list of items . . . “and combinations and mixtures thereof.”

Referenced herein are trade names for components including various ingredients utilized in the methods and kits described herein. The inventors herein do not intend to be limited by materials under a certain trade name. Equivalent materials (e.g., those obtained from a different source under a different name or reference number) to those referenced by trade name may be substituted and utilized in the descriptions herein.

The processes, methods, compositions, and apparatuses herein may comprise, consist essentially of, or consist of any of the features or embodiments as described herein.

In the description of the various embodiments of the present disclosure, various embodiments or individual features are disclosed. As will be apparent to the ordinarily skilled practitioner, all combinations of such embodiments and features are possible and can result in preferred executions of the present disclosure. While various embodiments and individual features have been illustrated and described, various other changes and modifications can be made without departing from the spirit and scope of the methods and kits described herein. As will also be apparent, all combinations of the embodiments and features taught in the foregoing disclosure are possible and can result in preferred executions.

Measuring

methods of assessing the health of animals are provided. More specifically, the methods relate to assessing the health of an animal by evaluating the performance of the animal subjected to a physical assessment. In specific embodiments, methods of measuring the activities of daily living of animals are provided. Also provided are methods relating to using an apparatus, subjecting an animal to a test on the apparatus, and making an assessment based on the results of the test.

Decrease in functional capacity with advancing age is well established in many animal species, including dogs. Loss of functional capacity can be associated with a decline in quality of life. Accordingly, it is envisioned that a performance assessment system can be utilized to translate this highly subjective area into an objective, consumer- observable assessment. In one very specific performance assessment system, a series of agility measures can be utilized to represent typical activities of a dog in a home environment. These typical activities as used herein are called Activities of Daily Living (ADL), as described in more detail hereinafter. A type of scoring system can be developed and utilized to evaluate the performance of the animal. In one way, the selection of tasks to be performed by the animal can be based on those activities that owners frequently report become increasingly more difficult for their animals to perform as they age. In one embodiment, dogs can be initially acclimated to the structures and tasks prior to the collection of any data. Data collections include an assessment of the dog's ability to complete each task and an assessment of the dog's ease to perform the task. Data can be collected over time and subsequently analyzed to assess the dog's ability to maintain functional activity over time and whether interventions can modulate any age-associated decline in activity. Such interventions can include dietary modification, supplement administration, weight loss/management plans, physical activity recommendations, veterinary intervention, and combinations thereof.

In particular embodiments, such performance assessments can be advantageous to comparing an animal's physical status before and after the consumption of a particular diet, especially in the case of a non-diseased animal. For example, some diets promote particular physical health benefits that will be experienced by the animal if the animal switches to and maintains a certain type or brand of food or a specific ingredient. An assessment that allows direct observation of particular physical health benefits of the animal could allow for the comparing of an animal's status before the consumption of the particular diet and after the animal switches to the particular diet. Additionally, such an assessment could be used as a supporting credentialing tool that supports particular health benefit claims that the particular diet is promoting. For example, if a particular diet is promoting that it will increase an animal's mobility, an assessment as disclosed herein can be used to support the specific health benefit claim to show that the companion animal's mobility has increased.

Non-limiting elements of animal health and physiology that could be benefited by diets include activity, mobility, joint mobility, weight loss, weight gain, excessive adipose tissue accumulation, anorexia, effects of aging, including mental awareness and activity levels, and ADL. Additional quality of life benefits that can be considered in animal health include, but are not limited to, the interaction of the individual with its environment such as behavior, temperament, companionship, social well-being, response to stress, cognition and sensorial abilities including vision, hearing, smell, taste, touch, and satiety, improved physical appearance such as physique, stature, body condition, skin condition, hair condition, and a more desirable social bond, such as the bond between a human and a companion animal, calculated indices of health, wellness, mobility, activity, vitality, frailty, functional living, health span, and active longevity.

Particular diets and animal health benefits can be those diets that include certain active components or ingredients. Some embodiments of those active components have been described in prior patent applications and can include such actives as Probiotics and calorie restriction mimetic ingredients such as mannoheptulose. Publications disclosing such active components or ingredients and health benefits include, U.S. Pat. No. 7,666,459, United States Publication Nos. 2005/0152884, 2005/0158293, 2005/0158294, 2005/0175598, and 2010/0003368, and WO05060707A2.

Thus, in one embodiment, the methods of assessment disclosed herein can be used as a tool to evaluate the effectiveness of diets positively impacting ADL. In another embodiment, the methods of assessment disclosed herein can be used as a credentialing or supporting tool for products, such as pet foods, that promote particular benefits. Therefore, the methods of assessment herein can be designed to translate the highly subjective area of a companion animals' health, such as physical status, into objective observation. In one embodiment, the methods of assessment herein guide the evaluations to be more objective, thus resulting in more reproducible data sets that are more amenable to statistical analysis, as shown herein.

In another embodiment, the method can provide perspective on whether the animal is suffering from particular types of mobility issues, such as bone issues and/or cartilage issues and/or joint issues and/or front v. hind leg issues. Such perspective can be based on the performance index of the animal as compared to the relevant performance profile, as described herein.

In another embodiment, the method can provide the ability to compare food products, breeds, ages, treatments, and combinations thereof. For example, the performance profiles as described herein can be used to assess animals after they have been exposed to food product to determine whether their performance profile as a group or their performance index individually has changed. Additionally, the performance profiles can be compared across breeds. Also, the performance profiles can be used to assess animals after they have been treated for particular issues to determine whether their performance profile as a group or their performance index individually has changed.

In another embodiment, the method can provide the ability to assess the change in the performance profile of a group of animals or the performance index of an individual animal over time. It can also be used to compare one animal to another animal of the same breed or different breeds. Thus, it can be used for monitoring animals over time and monitoring animals in groups. Additionally, the method can be used to monitor animals' performances within a group in which the animals of the group share at least one or more characteristics in common.

Activities of Daily Living (ADL)

Activities of Daily Living are generally activities that allow dogs to demonstrate their agility and ability to perform normal daily tasks. Exemplary normal daily tasks include climbing and descending stairs, walking throughout a home, jumping, running, getting into and out of a vehicle, performing trained activities, getting on and off furniture such as a bed or sofa, and generally being able to carry on a normal daily routine. Activity measurements may be designed to mimic common actions of dogs in a home environment which typically become more difficult to perform as animals age. Two methods are disclosed herein as being methods for assessing the performance of an animal with respect to ADL. Those two methods are Stairs and Rails and are disclosed in more detail hereinafter.

Other methods have been previously disclosed in publications. For example, jumping onto platform tables and jumping over hurdles. These were previously disclosed in United States Publication No. 2009/0253642. These methods can also be utilized for the methods of assessment as described herein.

Stairs

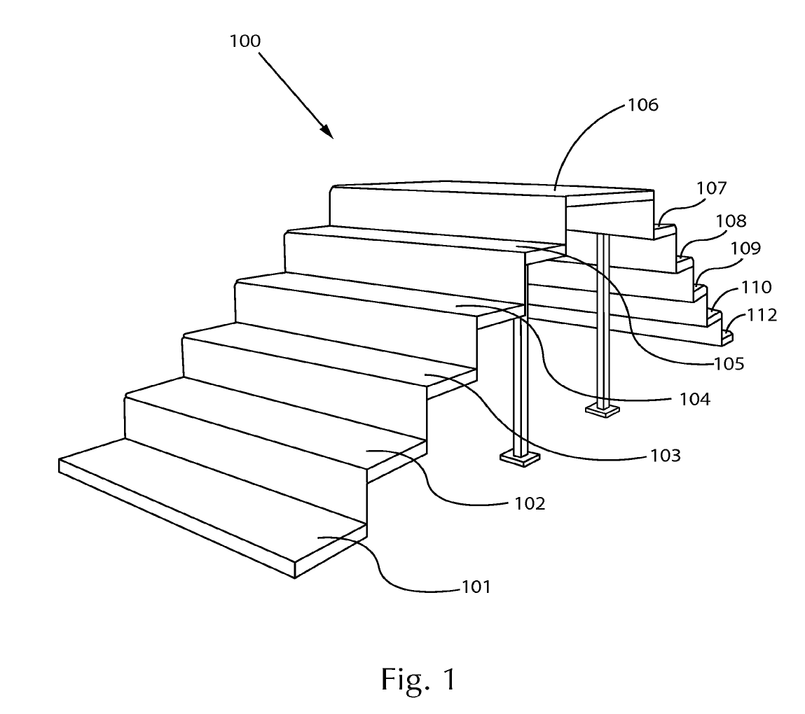

In one embodiment, a method of assessing the mobility, the activity, the capability, and/or the extent-to-be-able-to-move in an animal is provided. This method, in one embodiment, can include the use of an apparatus that is similar to a stairs arrangement. One specific embodiment of a stairs arrangement is shown in FIG. 1. Stairs arrangement 100 is provided. It should be understood that while the term “stairs” is used herein, that term is not meant to be limiting in any fashion, whether it be with respect to the number of platforms, the specific heights between platforms, or any other characteristic of stairs, whether or not shown in FIG. 1. Additionally, FIG. 1 only depicts one specific embodiment and is not meant as limiting in any fashion. A method of assessment by using a stairs arrangement can, in certain embodiments, provide observations related to many characteristics of a companion animal. For example, since a stairs arrangement can represent stairs in a home environment, it can provide observations regarding rehabilitation, neurological functions, range of motion (ROM), joint movement, laxity, coordination, balance, animal reliance on other things, usage of both hips and knees, musculoskeletal impact, and cardiovascular system function, among others.

As shown in FIG. 1, stairs arrangement 100 includes a top platform 106. Successive platforms that ascend to top platform 106 are provided. On the one side are platforms 101, 102, 103, 104, and 105 that lead to top platform 106. On the other side are platforms 107, 108, 109, 110, and 112 that lead to top platform 106. Each platform can have a corresponding height between it and the adjoining platform, as shown in FIG.

A method of using the stairs arrangement 100 for performance assessment is disclosed later herein.

Rails

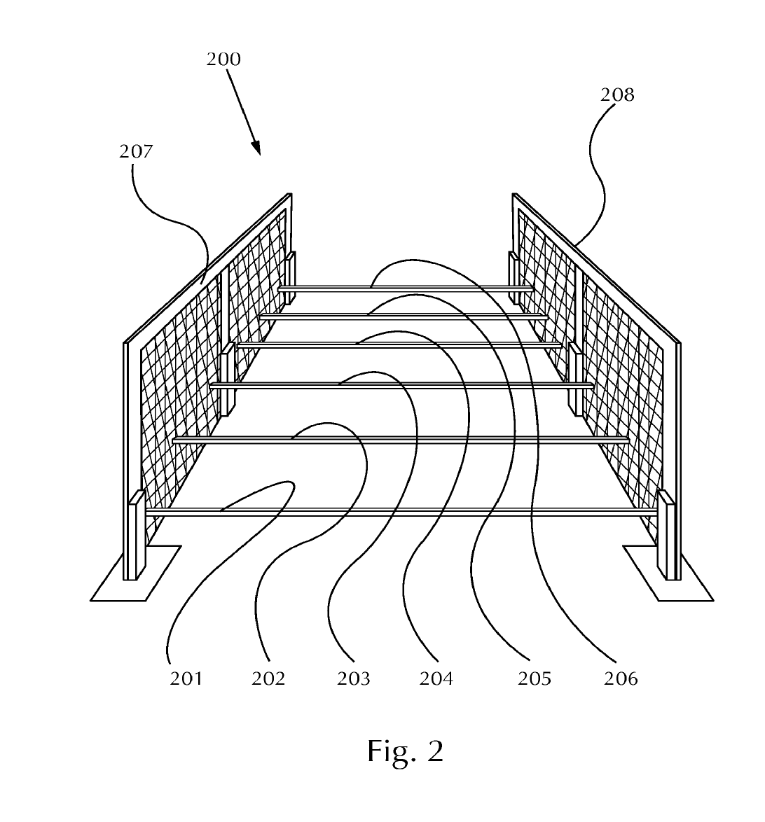

In one embodiment, a method of assessing the mobility, the activity, the capability, and or the extent-to-be-able-to-move in an animal is provided. This method, in one embodiment, can include the use of an apparatus that is similar to a rails arrangement. One exemplary embodiment of a rails arrangement is shown in FIG. 2. Rails arrangement 200 is provided. It should be understood that while the term “rails” is used herein, that term is not meant to be limiting in any fashion, whether it be with respect to the number of rails or hurdles, the specific distances between the hurdles, or any other characteristic of the hurdles, whether or not shown in FIG. 2. Additionally, FIG. 2 only depicts one specific embodiment and is not meant as limiting in any fashion. A method of assessment by using a rails arrangement can, in certain embodiments, provide observations related to many characteristics of a companion animal. For example, since a rails arrangement can represent stairs in a home environment, it can provide observations regarding mobility/agility, rehabilitation, neuromuscular function, range of motion (ROM), joint movement, laxity, flexion, extension, center of gravity, voluntary motor control, orthopedic function, neurological systems, stride, and elbow arthritis conditions, among others.

Rails arrangement 200 includes individual rails or hurdles 201, 202, 203, 204, 205, and 206, which are attached on their ends to supports 207 and 208. Each individual rail is spaced between the next individual rail, but the exact spacing is not necessarily critical. A given spacing or distance may be needed for a dog of a given size given the stride length and can be decided upon by those performing the assessment. The spacing or distance between each hurdle can be the same or different. The height of each hurdle can be the same or different than the height of the other hurdles. Accordingly, the distance between each hurdle and the height of each hurdle can each be adjusted.

A method of using the stairs arrangement 100 for performance assessment is disclosed later herein.

Methods

Activities of daily living (ADL) measurements can be collected utilizing activities such as Stairs, as shown in one embodiment in FIG. 1, and Rails, as shown in one embodiment in FIG. 2. These activities allow animals, such as dogs, to demonstrate their agility and ability to perform normal daily tasks. These activities and accompanying performance assessments and measurements thereof can be designed to mimic common actions of dogs in a home environment and which typically become more difficult to perform as dogs age.

Before data collection began, technicians train all dogs to perform the activities by using positive encouragement and reinforcement training techniques, such as clicker training. Such training and techniques are well known to those of ordinary skill in the art. Dogs are guided through each activity by a handler who uses encouragement devices (e.g., toys, diet kibbles as treats) and vocal praise. At no time does the handler touch the dog to provide assistance; however, due to the height of the stair obstacle, for example, a leash may be used to guide the dog for safety reasons.

Shoulder height of each individual dog is measured using a tape measure and recorded. Shoulder height is defined as the distance between the bottom of the right front leg (ground) to the proximal point of the scapular spine.

Stairs

The Stairs activity requires the dog to ascend and descend a set of custom designed stairs, one embodiment shown in FIG. 1, starting with the assent. The Rails activity requires the dog to maneuver over standard hurdles of a single height, as shown in FIG. 2. The height can be adjusted as appropriate, and can be a function of the shoulder height of the animal performing the test.

For both activities, dogs are scored for the successful completion or point of cessation in activity and ease of completion. For the Stairs, a time limit can be imposed. In one embodiment, a two-minute time limit is imposed. The ease of completion assessment is an indicator of how easily the dog performs the task. Ease of completion involves both age and/or pain-related difficulty, as well as motivation or reluctance and is scored on a 0-100 mm visual analog scale (VAS) ranging from “Very Difficult” to “Very Easy.”

In one specific embodiment, the Stairs activity is conducted indoors in a room equipped with the following equipment:

Aluminum stair structure constructed with six adjoining steps or platforms (111/2″ deep and 8″ high) up to the top platform (approximately 49″ in total height from floor, 48″ wide, 35″ across at the top), and six steps down. The structure is supported on aluminum legs with polyresin coating on landing surfaces and ¼″ polyboard backing behind each step.

Encouragement devices (e.g., toys, assigned diet kibbles for treats).

Leashes.

In some embodiments, the stairs are placed on gray matting to provide a non-slick and cushioned floor surface for the dogs. The mat (which may, for example, measure 3 feet width.times.15 feet length) provides some protective cushioning for the dogs as they ascend and descend the stairs. An additional shock-absorbing, anti-skid surface (such as one available from Top Dog Flooring; Ocala, Fla.; 81/2 mm thick with 10% gray color fleck; recycled tire rubber) can be used.

To perform the Stairs activity, the handler walks the dog on leash to the Stairs. The handler may lightly hold on to the leash during the activity and walk with the dog up and down, depending upon the dog. The handler makes a request of the animal to navigate the platforms. The dog is encouraged to ascend the stairs using vocal praise, toy, or treat. After ascending to the platform, the dog is encouraged to descend to the floor. The dog can ascend and descend the same side or up one side and down the other. The assessor records a “yes” if the dog successfully ascends and descends the structure. A “no” is recorded if the dog does not perform the task as described or makes no attempt to climb the structure. The time to completion is also recorded. The test is completed when the dog successfully completes the activity or receives a “no” score after a predetermined time limit, if a time limit is imposed. A time limit may, for example, be less than 5 minutes, less than 3 minutes, or less than 2 minutes. The time limit may be adjusted based on the specific obstacle set up (such as the number, height, or relative placement of the stairs or rails), the dog's health, the dog's age, and the like. If the animal does not fully complete the event, the following is recorded:

0—Dog refuses to conduct test due to physical reasons.

1—Front feet on steps.

2—All four feet on inclining steps.

3—Reaches top platform in entirety.

4—Front feet reach downward component to steps.

5—Completes stair test, returns to ground level without jumping or falling off

Many alternatives of the Stairs activity exist. For example, the stairs apparatus can have multiple platforms, for example a first platform, a second platform, a third platform, etc. The platforms can have differing heights such that a height change exists between the platforms, and the platforms can be arranged in any order such that a height change can be up or down between any of the platforms.

Rails

In one specific embodiment, the Rails activity is conducted indoors in a room equipped with the following:

Six rails made of PVC (½″ diameter, 52″ in length, and set 191/2″ apart) are secured in PVC lattice in stainless steel frames (24″ high by 120″ in length) enabling the rails to be set at a predetermined height of 6″ (measured from the floor to the top of the rail) make up the rails structure.

Turf spun roll, gray, 0.5 inches thickness, anti-fatigue matting.

Encouragement devices (e.g., toys, assigned diet kibbles for treats).

Leashes.

In this specific embodiment, the Rails structure is placed on gray matting to provide a non-slick and cushioned floor surface for the dogs. One or more mats (for example, two mats, each measuring 4.5 feet width.times.15 feet length, placed end-to-end or side-to-side) may be set under the structure. An additional shock-absorbing, anti-skid surface (such as one available from Top Dog Flooring; Ocala, Fla.; 81/2 mm thick with 10% gray color fleck; recycled tire rubber) can be used.

To perform the Rails activity, the handler makes a request of the animal to navigate over the hurdles by walking the dog on leash (if needed to guide the dog to stay on course) one way through the rails and the opposite way back. The dog gets two attempts in each direction to get a final score. The dog is encouraged by using vocal praise, toy, or treat. The handler does not touch the dog immediately before or during the activity. The handler may lightly hold on to the leash during the activity, depending upon the dog. To receive a successful score the dog must complete the course in no more than two attempts. The assessor records “yes” if the dog successfully completes the course. A score of “no” is recorded if the dog does not attempt the course. The number of attempts (one or two) is also recorded. The position of the dog's farthest front foot stops in each attempt is where completion is scored.

For either activity, dogs can be clicker trained. Clicker training is an animal training method based on behavioral psychology that relies on marking desirable behavior and rewarding it. Desirable behavior is marked by using a clicker, a mechanical device that makes a short, distinct ‘click’ sound, which tells the animal exactly when it is doing the right thing. This clear form of communication, combined with positive reinforcement, is an effective, safe, and humane way to teach an animal behavior(s) that the animal can physically and mentally perform. The human trainer clicks at the moment the behavior occurs, then delivers an appropriate reward (e.g., food, human interaction, toy). Once dogs are trained, the clicker may no longer be needed on a daily basis, however, rewards may be continued as positive reinforcement.

Scoring

As described above, the animal's performance may be assessed. The assessment is an indicator of how easily the dog performs the task. Ease of completion involves both age and/or pain-related difficulty, as well as motivation or reluctance and is scored on a 0-100 mm visual analog scale (VAS) ranging from difficult to easy for the animal to perform the activity to provide a VAS score. The VAS score is assigned by the handler based on a number of factors that the handler may observe based on the difficulty or ease of the animals performance of the activity. Such factors are based on descriptors that the handler observes, and some descriptors can be seen in both activities or just one of the activities. Those descriptors or attributes are well known in the art and can be broken down into descriptors that indicate difficulty and descriptors that indicate ease. The descriptors that can indicate difficulty include stiff movement; fixed movement; inability to clear poles; “bunny hop;” toe touching and/or dragging; very slow movements or just one step at a time; degree of hesitation, squatting prior to downward movement, for example; knuckling; difficulty landing; difficulty taking off, and circumduction (circling the leg such that the movement of the limb or extremity is such that the distal end describes a circle while the proximal end remains fixed or circular movement that combines flexion/extension with abduction/adduction, also described as circling the leg in the hip socket), as examples. The descriptors that can indicate ease include smooth movement; fluid movement; clears poles without hesitation; obvious placement of each leg; purposeful placement of each foot; running/fast completion; no hesitation/zest for performing; no noticeable knuckling; not difficulty with landing or taking off; normal joint movement, as examples. A multiplicity or aggregate of descriptors indicating difficulty results in a lower score.

The scoring can also be used for calibration of handlers, or those ones that are assessing the performance. For example, it may be desirable to train new handlers on the assessment of an animal's performance of the activities. To train the handler, it can be desirable to calibrate the handler by providing them with guide that will drive or direct the handler in the appropriate scoring methodology. To assist in calibration on the assessment, a method has been developed to evaluate the performance of the animals to drive the objectivity of the assessment.

In one embodiment, the method includes providing a first group of companion animals, the first group comprising a first plurality of companion animals; subjecting each of the first plurality of companion animals to a performance assessment; evaluating a performance of each companion animal based on a plurality of attributes, wherein the attributes are associated with the health of the companion animal, wherein the evaluating comprises: providing a performance profile; accounting for the attributes exhibited by each companion animal during the performance; assigning a performance index based on the performance profile given the attributes accounted for.

As described above, the performance assessment can be the activities as described herein. Subjecting the animals to the activity can then be performed by the handler/assessor. As the animal is subjected to the activity, the handler can evaluate the performance of the animal based on the descriptors or attributes as described above. The attributes indicate the level of ease and/or difficulty that the animal has in performing the activity and thus are associated with the health of the companion animal and specifically the mobility, activity, and capability of the animal. The evaluation by the handler can then include accounting for the number of negative or difficulty attributes exhibited by the animal during the performance of the activity.

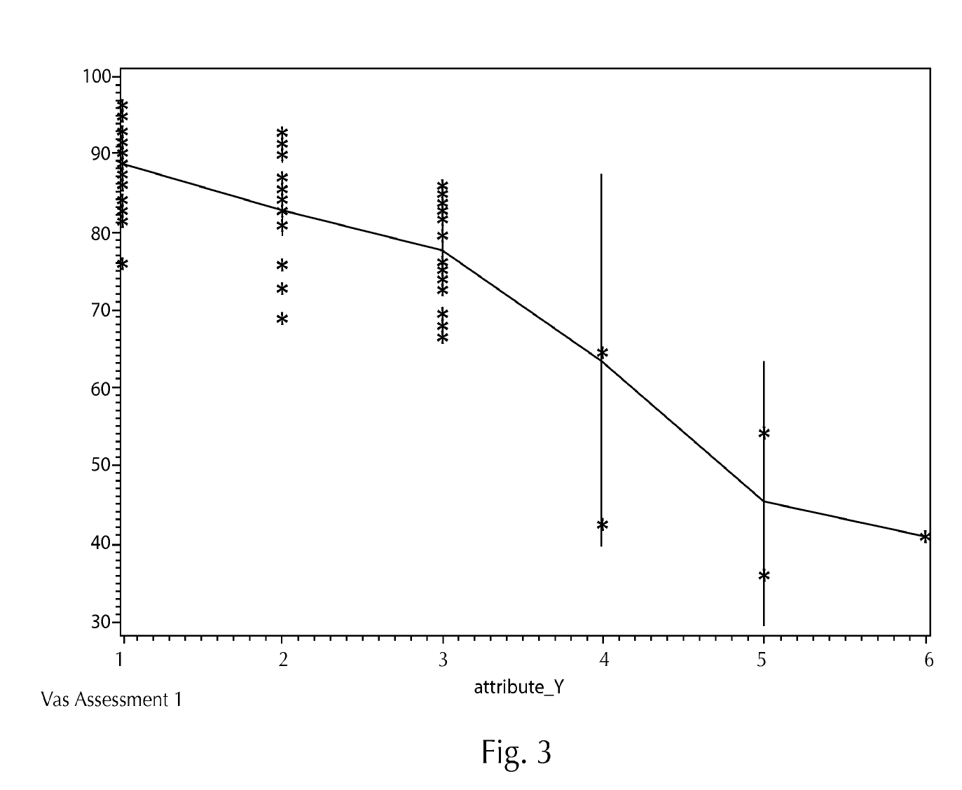

A performance profile can be provided that can then guide the subjectivity. The performance profile can be based on a database of past performances such that the profile is a compilation of past performance assessments. Examples of performance profiles are shown in FIGS. 3, 4, and 5. FIG. 3 shows one compilation of scores for the Rails activity. The compilation of scores is based on scores of evaluations of assessments. The x-axis represents the number of negative or difficulty performance attributes that are observed during the performance of the activity. The y-axis represents the VAS score assigned to the animal's performance for the specific number of attributes observed. For example, in a performance in which only one negative attribute was observed for an animal for the Rails activity, the VAS scores assigned by the assessor ranged from about 76 to about 96. In a performance in which exactly two negative attributes were observed for an animal for the Rails activity, the VAS scores assigned by the assessor ranged from about 68 to about 93. The VAS score range for three attributes, four attributes, five attributes, and six attributes is similarly shown in FIG. 3. As expected, for an increasing number of negative attributes observed, the general trend is for the VAS score to decrease, which is to be expected because as an assessor observes more negative attributes, the VAS score is expected to decrease.

Based on the VAS scores for the attributes, a performance profile can be constructed. The performance profile can be constructed in any one of several ways. For example, an average of the scores can be constructed such that when a later performance is assessed, a performance index can be assigned that is the average for that specific number of attributes observed. As shown in FIG. 3, for three attributes observed, the average VAS score assigned is about 78. Therefore, in any later performance that is assessed, and if three attributes are observed, then a VAS score or performance index of 78 is assigned to that animal's performance. However, other embodiments of performance profiles can be constructed. In one embodiment, a band can be constructed. A band can be constructed that provides an upper and lower limit for each attribute accounted for. Thus, if three attributes are assessed, the profile might provide a range of about 66 to about 92 for the performance index. This upper and lower limit for each number of attribute provides a band of scores that may indicate level of severity of the attributes observed. For example, if the attributes observed were all severe, then possibly the lower of the band indices is selected for the performance index. It the attributes observed were not severe, then possibly the higher of the band indices is selected for the performance index.

Other embodiments in addition to the average and the band are envisioned. Specific, rigid maximum and minimum limits can be imposed. Only a maximum limit can be imposed. Alternatively, only a minimum limit could be imposed. Any of the performance profiles can be provided as graphs or tables.

Accordingly, performance profile can be constructed and leveraged for assigning a performance index for the performance of the animal based on the number of attributes observed. These performance profiles thus provide improved objectivity for assigning the proper performance index to the animal's performance, which can allow for easier training and calibration of assessors. For example, if an assessor notices three attributes, then they are already guided to the performance profile from which to use for assigning the performance index.

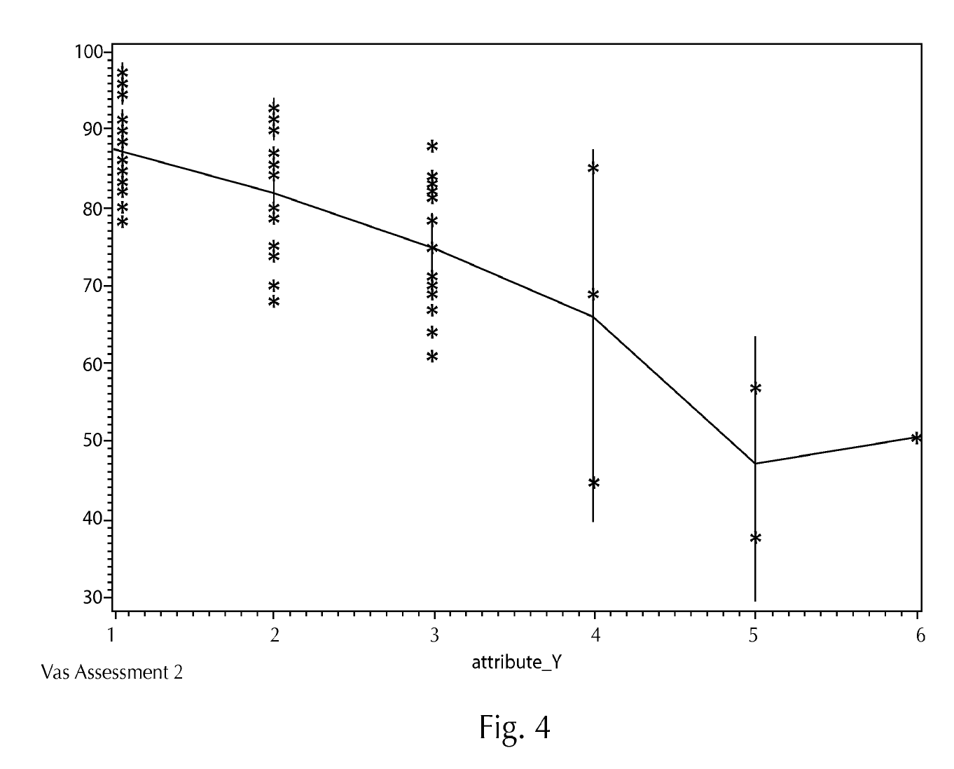

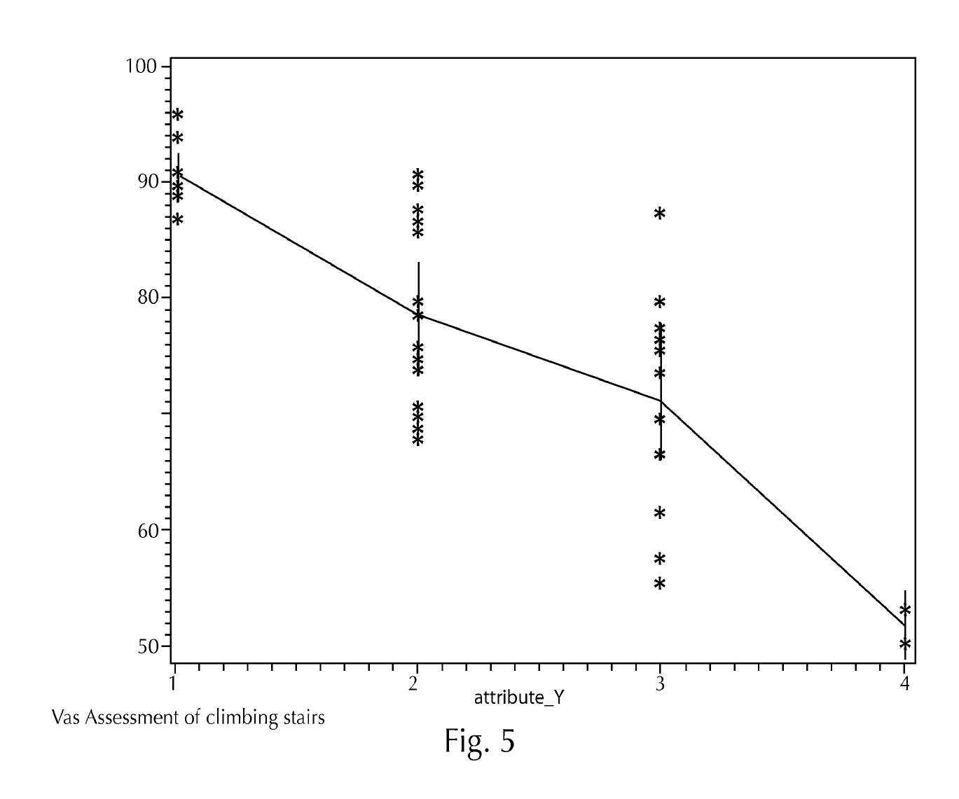

Additional graphs of performance profiles are shown in FIG. 4 and FIG. 5. FIG. 4 represents a graph of a second performance profile for the Rails, which is in addition to the graphs of the performance profile in FIG. 3. FIG. 5 represents a graph of a performance profile for the stairs. The description above with respect to FIG. 3 is equally applicable to these additional figures.

It should be noted that while it has been described that the handler performs the assessment, another embodiment includes that a separate assessor of the performance can be present to provide the evaluation. In such an embodiment, the handler can request the assessment while the assessor evaluates the performance.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any embodiments disclosed or claimed herein or that it alone, or in any combination with any other reference or references, teaches, suggests or discloses any such embodiment. Further, to the extent that any meaning or definition of a term in this document conflicts with any meaning or definition of the same term in a document incorporated by reference, the meaning or definition assigned to that term in this document shall govern.

While particular embodiments have been illustrated and described, it would be obvious to those skilled in the art that various other changes and modifications can be made without departing from the spirit and scope of the described kits and methods. It is therefore intended to cover in the appended claims all such changes and modifications that are within the scope of this description.

What is claimed is:

1. A kit comprising:
A pet food formulated to provide a health benefit;
A credentialing tool comprising a performance profile; and
Instructions for feeding the food and assessing the impact thereof on the health of the pet using the credentialing tool, Wherein the performance profile comprises a compilation of past performance assessments of at least one activity for pets of a similar age, size and/or breed.

2. The kit of claim 1, wherein the credentialing tool further comprises a guide describing appropriate scoring methodology for the at least one activity.

3. The kit of claim 1, wherein the performance profile is provided in the form of a database, graph or table.

4. A method for assessing the impact of a diet on a pet comprising Feeding the pet the diet;
Having the pet perform at least one activity; and
Assessing the impact of the diet by comparing the pet's performance of the at least one activity to a performance profile comprising a compilation of performance assessments of the at least one activity for pets of a similar age, size and/or breed.

5. The method of claim 4, further comprising assigning a performance index to the pet's performance of the at least one activity.

6. The method of claim 5, further comprising having the pet perform the at least one activity multiple times and comparing the performance indices for each assessment.

7. The method of claim 6, further comprising having the pet perform the at least one activity prior to feeding the pet the diet.
